(19) United States
(12) Patent Application Publication
(10) Pub. No.: US 2008/0245372 A1
(43) Pub. Date: Oct. 9, 2008

(54) REUSABLE FOAM EAR INSERT

(76) Inventor: Richard C. Smith, Costa Mesa, CA (US)

Correspondence Address: MACPHERSON KWOK CHEN & HEID LLP, 2033 GATEWAY PLACE, SUITE 400, SAN JOSE, CA, 95110, US

(21) Appl. No.: 11/869,526

(22) Filed: Oct. 9, 2007

Related U.S. Application Data
(63) Continuation-in-part of application No. 11/411,314, filed on Apr. 26, 2006.
Application No. 11/411,314 is a continuation-in-part of application No. 11/247,105, filed on Oct. 11, 2005.

(51) Int. Cl. A61F 11/10

(52) U.S. Cl. 128/864

(57) ABSTRACT

A reusable foam ear insert is disclosed. The ear insert can comprise a foam body and a non-foam covering. The foam body can, for example, comprise foam rubber. The covering can, for example, comprise vinyl. The body is configured to be at least partially received within the ear canal and can be generally bullet shaped. The combination of the body and the covering can provide hearing protection. An optional bore can facilitate use in communications. The covering is resistant to soiling and is easily cleaned, thus facilitating reuse of the ear insert.

### RELATED APPLICATIONS

This patent application is a continuation-in-part (CIP) of U.S. patent application Ser. No. 11/411,314, filed on Apr. 26, 2006, and entitled EARPIECE WITH EXTENSION (docket no. M-15744-1P US); which is a continuation-in-part (CIP) of U.S. patent application Ser. No. 11/247,105, filed on Oct. 11, 2005, and entitled EARPIECE WITH FLANGED EXTENSION (docket no. M-15744 US), the entire contents of both of which are hereby expressly incorporated by reference.

### TECHNICAL FIELD

The present invention relates generally to ear inserts. The present invention relates more particularly to reusable ear inserts for use in hearing protection, communications, listening to music, and the like.

### BACKGROUND

Foam inserts for hearing protection are well known. Such foam inserts are typically compressed and then inserted into the ear canal. Foam inserts can be compressed by rolling them between the fingers so as to temporarily reduce the diameter thereof. The foam expands to conform to the ear canal after it has been inserted therein.

Foam inserts are used to protect the ears from loud noises, such as those that result from firearms, heavy machinery, and jet engines. Foam inserts reduce the noise level of sound traveling through the user's ear canal. Their use tends to mitigate the undesirable occurrence of ear problems such as hearing loss and tinnitus.

Although such contemporary foam ear inserts have proven generally suitable for their intended purposes, they possess inherent deficiencies which detract from their overall effectiveness and desirability. For example, contemporary foam ear inserts are generally used only once and then discarded. Such practice is costly and wasteful.

### BRIEF SUMMARY

A reusable foam ear insert is disclosed. The reusable foam ear insert can be used for hearing protection and/or communications. The ear insert can comprise a body and a covering, wherein the body is substantially more compressible than the covering and the covering is smoother than the body.

For example, the ear insert can comprise a foam body and a non-foam covering. More particularly, the body can comprise foam rubber and the covering can comprise vinyl. Those skilled in the art will appreciate that other materials are likewise suitable.

The body can be configured to be at least partially received within the ear canal and can be generally bullet shaped. The combination of the body and the covering can provide hearing protection. Thus, the body and the covering cooperate to define ear plugs.

An optional bore formed through the body can facilitate use in communications. Thus, a communications cable assembly can be defined. The communications cable assembly can comprise an ear insert, acoustic tubing in acoustic communication with the bore, and a speaker in acoustic communication with the acoustic tubing. The acoustic tubing can be small diameter acoustic tubing. For example, the acoustic tubing can have a diameter of approximately 0.0455 inch.

An optional bore formed through the body can also be formed so as to provide pressure relief. As those skilled in the art will appreciate, the insertion of ear inserts into the ear canal, particularly when the ear inserts are being used as earplugs, is often accompanied by an increase in pressure within the ear canal. This increase in pressure can be uncomfortable. The bore can relieve the pressure whether the ear inserts are being used for hearing protection or for communications. The bore can relieve the pressure by permitting air from the ear canal to travel therethrough.

The reusable foam ear insert can be made by at least partially covering a foam body with a non-foam material. Various methods can be used to cover the foam body with the non-foam material. For example, the foam body can be dipped into a liquid that forms the non-foam material upon the body as the liquid cures.

The reusable foam ear insert is comfortable to wear, particularly for extended periods of time. The covering is resistant to soiling and is easily cleaned, thus facilitating reuse of the ear insert.

This invention will be more fully understood in conjunction with the following detailed description taken together with the following drawings.

### BRIEF DESCRIPTION OF THE DRAWINGS

FIG. 1 is a semi-schematic perspective view of a reusable foam ear insert for hearing protection, according to an exemplary embodiment of the present invention;

FIG. 2 is a semi-schematic cross-sectional side view of the reusable foam ear insert of FIG. 1, showing the foam body and the smooth covering formed thereover;

FIG. 3 is a semi-schematic perspective view of a reusable foam ear insert for communications, listening to music, and the like according to an exemplary embodiment of the present invention;

FIG. 4 is a semi-schematic cross-sectional view of the reusable foam ear insert of FIG. 3, showing the foam body, the smooth covering formed over the foam body, and the bore formed longitudinally therethrough;

FIG. 5 is a semi-schematic rear view of a portion of a communications cable assembly having a reusable foam ear insert attached to the acoustic tubing thereof, according to an exemplary embodiment of the present invention;

FIG. 6 is a semi-schematic side view of the portion of a communications cable assembly of FIG. 5;

FIG. 7 is a semi-schematic side view of the portion of a communications cable assembly having a reusable foam ear insert attached to the acoustic tubing thereof, according to another exemplary embodiment of the present invention;

FIG. 8 is a semi-schematic end view of an earpiece;

FIG. 9 is a semi-schematic side view of the earpiece of FIG. 8;

FIG. 10 is a semi-schematic end view of the earpiece of FIG. 8 having a reusable foam insert attached thereto, according to another exemplary embodiment of the present invention;

FIG. 11 is a semi-schematic side view of the earpiece and reusable foam insert of FIG. 10, show a barbed fitting attaching the earpiece to the reusable foam insert; and

FIG. 12 is a semi-schematic side view of the portion of the communications cable assembly of FIG. 6 having the earpiece of FIG. 11 attached thereto, according to another exemplary embodiment of the present invention.

Embodiments of the present invention and their advantages are best understood by referring to the detailed description that follows. It should be appreciated that like reference numerals are used to identify like elements illustrated in one or more of the figures.

### DETAILED DESCRIPTION

Contemporary foam ear inserts are typically discarded after each use because they become soiled during use. Such soiling is the result of earwax, dirt, and/or other matter accumulating thereon. The surface of foam is particularly susceptible to such soiling due to the texture thereof. The many small pores of the foam surface trap and hold debris in the same fashion that a household sponge does.

Although solid rubber ear inserts do not tend to accumulate debris and are easily cleaned, solid rubber ear inserts tend to be less comfortable than foam inserts. Indeed, solid rubber ear inserts can harm the tissue of the ear canal. This is particularly true when the solid rubber ear inserts are worn for extended periods of time.

Reusable foam ear inserts are disclosed. The reusable foam ear inserts can comprises a substantially smooth skin or covering formed over a foam body. The covering tends to prevent the ear insert from accumulating debris in much the same manner as the smooth outer surface of a solid rubber ear insert. The foam body tends to provide the comfort of a contemporary foam ear insert.

Such reusable foam ear inserts can be reused many times because they do not tend to become soiled and because when they do become soiled, they can easily be cleaned. Generally the reusable foam ear inserts can be cleaned simply by wiping any debris therefrom with a tissue or cloth. Soap and water can be used to remove more stubborn soiling.

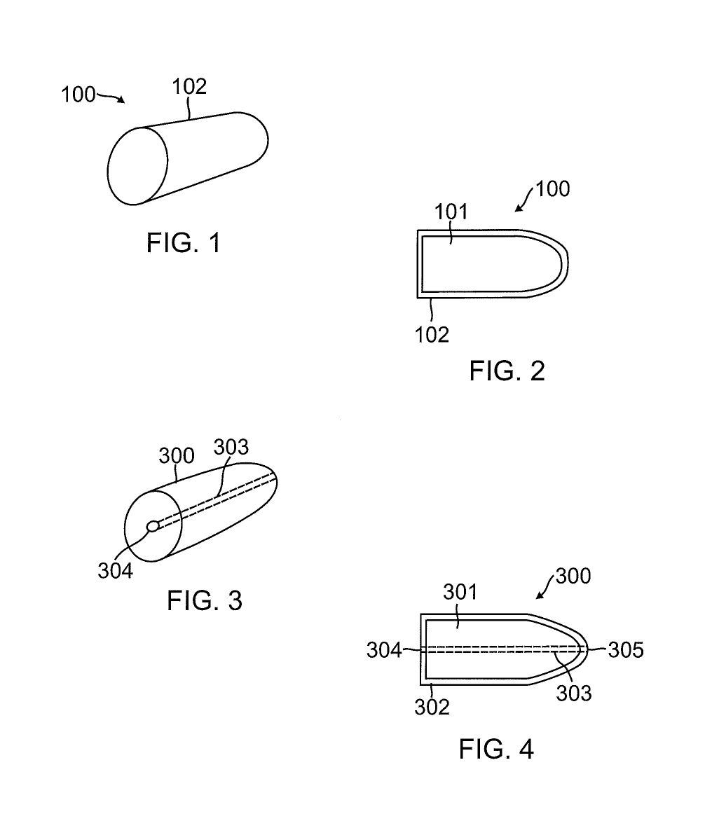

Referring now to FIG. 1, an exemplary embodiment, the reusable foam ear insert 100 can be generally cylindrical and can have one (or two) pointed or rounded ends. Thus, the reusable foam ear insert 100 can be generally bullet shaped. Those skilled in the art will appreciate that other shapes are likewise suitable. For example, the reusable foam insert 100 can have generally flat ends and thus be more cylindrical in shape.

Referring now to FIG. 2, the reusable foam ear insert 100 can comprise a foam body 101. The foam body 101 can be generally bullet shaped, as described above. The foam body 101 can have a substantially smooth covering 102 formed completely thereover.

Those skilled in the art will appreciate that other configurations are likewise suitable. For example, the cover 102 can be formed over only a potion or portions of the body 101. The body 101 can comprise a non-foam core, such as a core of rubber or the like.

The foam core 101 can comprise foam rubber or the like. The foam rubber can be a silicon foam rubber. The covering 102 can comprise vinyl, for example. Generally, the covering 102 can be biocompatible, such that it does not irritate or damage the ear. Those skilled in the art will appreciate that other materials are likewise suitable.

The body 101 can be slightly larger in diameter than the ear canal. The body 101 can be approximately the size of a contemporary foam ear insert. The covering 102 can be between 0.001 inch and 0.020 inch thick, for example. Other thicknesses are likewise suitable.

The covering 102 can be applied to the body 101 by dipping the body 101 into a liquid comprising the covering material. Alternatively, the covering 102 can be applied to the body 101 by spraying, adhesive bonding, or by any other desired method.

The covering 102 can be formed integrally with the body 101, such as from the same material. This can be accomplished during the molding process, for example. Thus, the foam material can be injected or otherwise formed in a mold in a manner that causes the outer surface thereof to form a generally smooth covering.

Optionally, a leash or lanyard can be attached to the reusable ear insert. For example, a single lanyard can attach two reusable ear inserts together. Alternatively, each reusable ear insert can have a dedicated lanyard.

The reusable ear insert 100 of the present invention can be used in generally the same manner as a contemporary foam ear insert. It can be compressed by rolling between the fingers and then placed into the ear canal. Inside of the ear canal, the reusable ear insert will expand to fit the ear.

Since the reusable ear insert 100 of the present invention comprises a foam body, it is comfortable to wear. Since the reusable ear insert 100 of the present invention comprises a substantially smooth covering 102, it does not tend to become soiled as readily as does a contemporary foam ear insert and it can be readily cleaned if it does become soiled.

Further, hearing protection can be enhanced by the use of the covering 102. The covering 102 provides an additional barrier to ambient sound that thus reduces the level of sound traveling through the ear canal.

The smooth covering 102, 302 can make insertion of the ear insert 100, 300 into the ear canal easier. The smooth covering 102, 302 can also make adjustment of the position of the ear insert 100, 300 easier after it has been inserted into the ear canal.

Referring now to FIGS. 3 and 4, another exemplary embodiment, the reusable foam ear insert 300 can comprise a foam body 301 having a covering 302 formed thereover as discussed above and additionally having a bore 303 formed generally centrally and longitudinally therethrough. The bore can be formed concentrically with respect to the central axis of the body 301 or can be formed eccentrically with respect thereto. The bore 303 can have an outer (generally disposed outside of the ear) opening 304 and an inner (generally disposed inside of the ear) opening 305.

This embodiment of the present invention can be used for communications, listening to music, and the like. The bore 304 facilitates the communication of sound through the ear canal. As an example of use in communications, the reusable foam ear insert can be attached to a cable assembly to facilitate use with a two-way radio such as those used by police, firemen, and the military. As a further example of use in communications, the reusable foam ear insert can be used with an earpiece for a cellular telephone. As an example of use for listening to music, the reusable foam ear insert can be used with an MP3® player, an iPod®, a personal computer, or the like.

When used in communications and/or listening to music, sound can be provided to the bore 303 by a speaker. For example, sound can be provided to the bore 303 by a speaker via the use of acoustic tubing as discussed below. Sound can also be provided to the bore 303 by other means, such as by a speaker attached directly to the ear insert (without using acoustic tubing).

The embodiment of the ear insert shown in FIGS. 3 and 4 can be used in a communication system wherein the bore 303 facilitates the passage of sound through the ear canal. The embodiment of the ear insert shown in FIG. 3 and 4 can also be used for hearing protection. In this instance, the bore 303 can provide pressure relief by allowing pressure formed in the ear canal when the ear insert is inserted to bleed off. The bore 303 can also allow some ambient sound to be heard while the ear insert mitigates sound generally.

The ear insert 300 can be configured so as to selectively attenuate sounds having some frequencies and so as to selectively transmit sounds having other frequencies. For example, the bore 303 can be configured so as to selectively pass voice while the body selectively attenuates loud noises from machinery, gunshots, jet engines and the like. A filter, such as a Hochs filter, can be used to facilitate such selectivity.

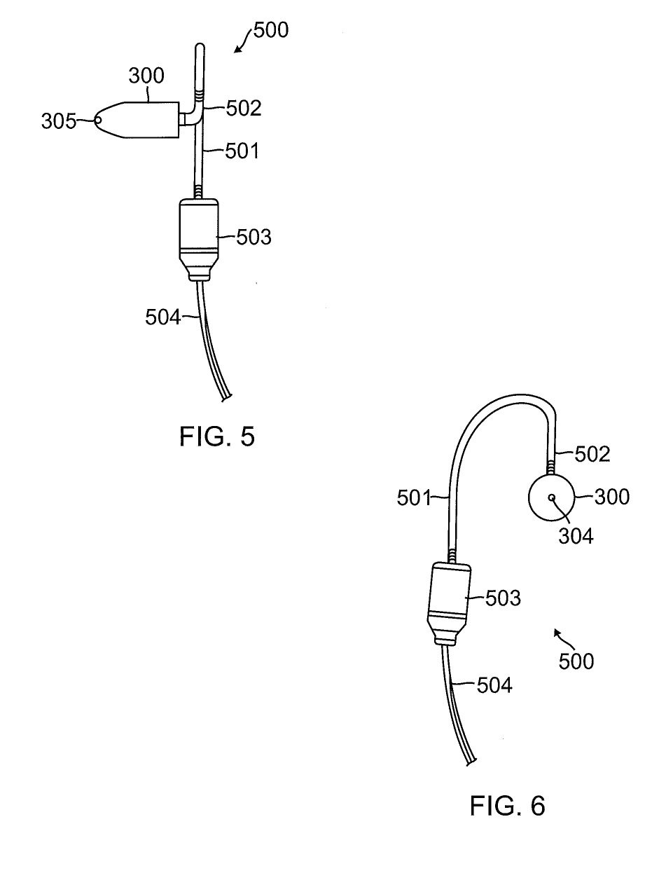

Referring now to FIGS. 5 and 6, a communications cable assembly 500 can comprise a reusable foam ear insert, such as insert 300 of FIGS. 3 and 4. One end of barbed elbow fitting 502 can be inserted through opening 304 into the bore 303 of the insert 300. The elbow fitting 502 can extend substantially into or completely through the bore 303. By having the elbow fitting 502 extend completely through the bore 303, the bore 303 may be better prevented from collapsing when the insert 300 is inserted into the ear canal.

The elbow fitting 502 can extend almost completely through the bore 303 such that the likelihood of the bore 303 collapsing is mitigated and such that the inner end of the elbow fitting 502 does not protrude from the insert 300. In this manner, the inner end of the elbow fitting 502 is cushioned and, consequently, the likelihood of the inner end of the elbow fitting 502 undesirably contacting the ear canal, such as during insertion of the insert 300, is mitigated.

The other end of the elbow fitting 502 can be attached to acoustic tubing. The elbow fitting 502 can be attached to standard diameter acoustic tubing, such as acoustic tubing having an outer diameter of approximately 0.125 inch. Alternatively, the elbow fitting 502 can be attached to small diameter acoustic tubing, such as acoustic tubing having a diameter of less than approximately 0.060 inch. For example, the elbow fitting 502 can be attached to small diameter acoustic tubing having an outer diameter of approximately 0.0455 inch.

The use of small diameter acoustic tubing tends to make the wearing of a communications cable assembly less conspicuous. Making the wearing of a communications cable assembly less conspicuous can be beneficial in applications such as covert police and security use.

The acoustic tubing 501 can be attached to a speaker 503. The speaker 503 can be in electrical communication with a two-way radio, an MP3 player, an iPod, a personal computer, a cellular telephone, or the like via electrical cable 504.

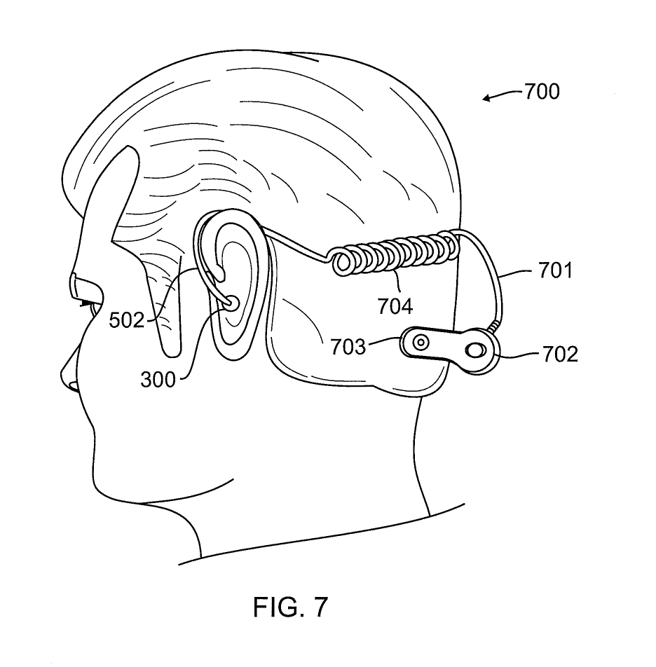

Referring now to FIG. 7, another embodiment of the communications cable assembly is shown as it is being worn by a user. The ear insert 300 is positioned in the user's ear canal. Small diameter acoustic tubing 701 extends from the ear insert 300 to a speaker 702. The speaker 702 can attach to the user's collar, such as via a clip 703. Electrical cable 504 can provide electrical communication between the speaker 702 and a two-way radio or other device as discussed above.

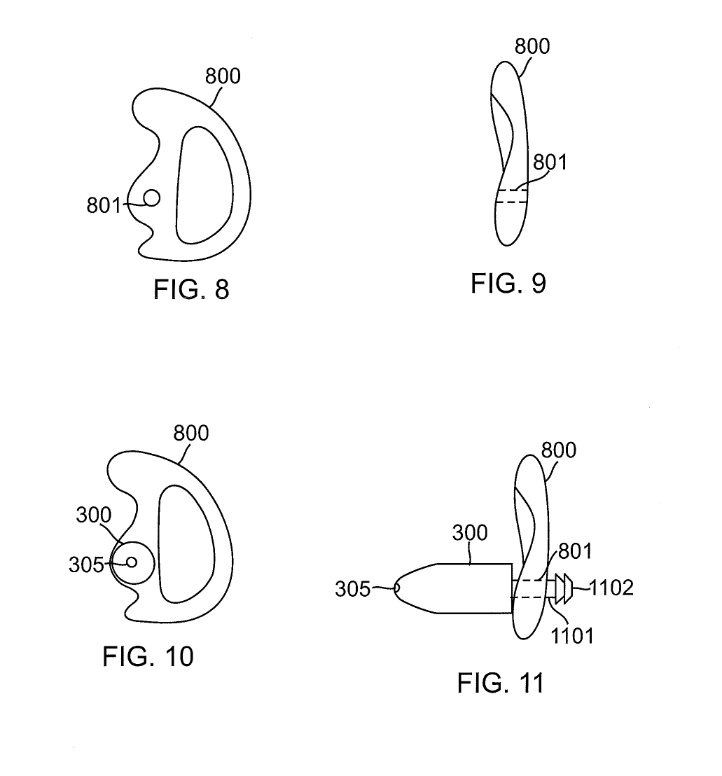

Referring now to FIGS. 8 and 9, an earpiece 800 suitable for use with one or more embodiments of the present inventions is shown. The earpiece can be similar to the EP1 and EP2 earpieces sold by SureFire of Fountain Valley, Calif. The boss (that portion that extends into the ear canal) of SureFire's EP1 earpiece can be removed and a larger hole 801 can be provided, like SureFire's EP2.

Referring now to FIG. 10 and 11, an earpiece assembly can comprise an optional earpiece 800 (such as that of FIGS. 8 and 9). The earpiece 800 can fit into the conchae of a user's ear and hold the foam insert 100, 300 in the user's ear canal. The foam insert 100, 300 can be used either with or without an earpiece.

The earpiece 800 can be attached to the foam insert 100, 300 by any suitable means. For example, a fitting 1101 can extend through the hole 801 of earpiece 800 and into the foam insert 300. The fitting can be solid for use in earplugs or the fitting can be hollow (so as to define a tube) and can have a barbed outer end 1102 for use in communications (to permit sound to travel though the earpiece 800 and the foam insert 300.

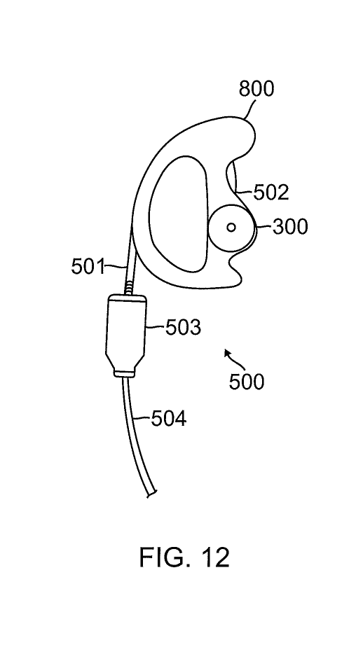

Referring now to FIG. 12, the communications cable assembly of FIG. 6 is shown with the optional earpiece 800 attached thereto so as to better hold the foam ear insert 300 in the ear canal.

Although the reusable foam ear insert 300 of FIGS. 3-7 and 10-12 is intended for use in a communications system, the reusable foam ear insert 300 can provide at least some hearing protection. The reusable foam ear insert 300 inherently reduces the level of ambient sound traveling through the ear canal.

Thus, the reusable foam ear insert of the present invention enhances comfort while allowing it to be reused. Reuse of the foam ear insert substantially reduces the costs associated therewith and also reduces undesirable waste.

Embodiments described above illustrate, but do not limit, the invention. It should also be understood that numerous modifications and variations are possible in accordance with the principles of the present invention. Accordingly, the scope of the invention is defined only by the following claims.

1 An ear insert comprising a foam body and a non-foam covering.

2. The ear insert as recited in claim 1, wherein the body comprises foam rubber.

3. The ear insert as recited in claim 1, wherein the non-foam covering comprises a material that is substantially smoother than the body.

4. The ear insert as recited in claim 1, wherein the non-foam covering comprises vinyl.

5. The ear insert as recited in claim 1, wherein the body is generally cylindrical.

6. The ear insert as recited in claim 1, wherein the body comprises a generally flat end and a generally rounded end.

7. The ear insert as recited in claim 1, wherein the body is generally bullet shaped.

8. The ear insert as recited in claim 1, wherein the body comprises a bore through which sound can travel within the ear canal.

9. The ear insert as recited in claim 1, wherein the body comprises a bore through which sound can travel within the ear canal, the bore being sized to receive tubing having a outer diameter of approximately 0.0455 inch.

10. An ear insert comprising a body and a covering, the body being substantially more compressible than the covering and the covering being smoother than the body.

11. A communications cable assembly comprising:
an ear insert comprising a foam body, a non-foam covering, and a bore extending through the body;
acoustic tubing in acoustic communication with the bore; and
a speaker in acoustic communication with the acoustic tubing.

12. The communications cable assembly as recited in claim 11, wherein the acoustic tubing has an outer diameter of less than 0.060 inch.

13. The communications cable assembly as recited in claim 11, wherein the acoustic tubing has an outer diameter of approximately 0.0455 inch.

14. The communications cable assembly as recited in claim 11, wherein the body comprises foam rubber.

15. The communications cable assembly as recited in claim 11, wherein the non-foam covering comprises vinyl.

16. A method for making an ear insert, the method comprising at least partially covering a foam body with a non-foam material.

17. The method as recited in claim 16, wherein the body is configured for hearing protection.

18. The method as recited in claim 16, wherein the body is configured for communications.

19. The method as recited in claim 16, wherein the body comprises foam rubber.

20. The method as recited in claim 16, wherein the covering comprises vinyl.

21. An earpiece assembly can comprise:
ear insert comprising a foam body and a non-foam covering; and
an earpiece attached to the ear insert.

22. The earpiece assembly as recited in claim 21, further comprising a fitting attaching the earpiece to the ear insert.

23. The earpiece assembly as recited in claim 21, further comprising a solid fitting attaching the earpiece to the ear insert such that the earpiece assembly is suitable for use as an earplug.

24. The earpiece assembly as recited in claim 21, further comprising a hollow barbed fitting attaching the earpiece to the ear insert such that the earpiece assembly is suitable for use in communications.
